(19) United States
(12) Patent Application Publication
(10) Pub. No.: US 2015/0265535 A1
(43) Pub. Date: Sep. 24, 2015

(54) SUSTAINED-RELEASE LIPID PRE-CONCENTRATE OF CATIONIC PHARMACOLOGICALLY ACTIVE SUBSTANCE AND PHARMACEUTICAL COMPOSITION COMPRISING THE SAME

(71) Applicant: CHONG KUN DANG PHARMACEUTICAL CORP., Seoul (KR)

(72) Inventors: Ha Na Yu, Gyeonggi-do (KR); Hye Jung Baik, Gyeonggi-do (KR); Won Kyu Yang, Gyeonggi-do (KR); Jin Young Ko, Gyeonggi-do (KR); Sung Bum Jung, Gyeonggi-do (KR); Sung Won An, Gyeonggi-do (KR); Min Hyo Ki, Gyeonggi-do (KR)

(21) Appl. No.: 14/440,058

(22) PCT Filed: Dec. 27, 2013
(86) PCT No.: PCT/KR2013/012259

§ 371 Date: Apr. 30, 2015

(30) Foreign Application Priority Data
Dec. 28, 2012 (KR) 10-2012-0157562

(51) Int. Cl. A61K 9/127; A61K 31/522; A61K 47/14; A61K 47/24; A61K 47/20; A61K 38/26; A61K 47/26; A61K 47/12; A61K 31/519; A61K 31/4196; A61K 38/21; A61K 38/08; A61K 47/28

(52) U.S. Cl. CPC A61K 9/1274; A61K 38/08; A61K 31/522; A61K 47/14; A61K 47/24; A61K 47/20; A61K 47/28; A61K 47/26; A61K 47/12; A61K 31/519; A61K 31/4196; A61K 38/21; A61K 38/26

(57) ABSTRACT

Disclosed is a sustained-release lipid pre-concentrate, comprising: a) at least one liquid crystal former; b) at least one neutral phospholipid; c) at least one liquid crystal hardener; and d) at least one anionic anchoring agent, wherein the sustained-release pre-concentrate exists as a lipid liquid phase in the absence of aqueous fluid and forms into a liquid crystal upon exposure to aqueous fluid. The sustained-release lipid pre-concentrate is configured to enhance the sustained release of cationic pharmacologically active substance through ionic interaction between the anionic anchoring agent and the cationic pharmacologically active substance.

### TECHNICAL FIELD

The present invention relates to a sustained release lipid pre-concentrate comprising an anionic anchoring agent, and a pharmaceutical composition comprising the same.

### BACKGROUND ART

Sustained release formulation is designed to release a single dose of a pharmacologically active substance at a predetermined rate in order to maintain the effective concentration of the substance in the blood stream for a specific period of time, with minimization of the side effects caused by multiple doses.

PLGA [poly(lactic-co-glycolic acid)] is a representative of the currently used biodegradable materials which are approved for use in sustained release by the Food and Drug Administration (FDA). U.S. Pat. No. 5,480,656 describes the sustained release of a pharmacologically active substance by way of the degradation of PLGA into lactic acid and glycolic acid over a specific period of time in vivo. However, the acidic degradation products of PLGA induce inflammation, decreasing cell growth (K. Athanasiou, G. G. Niederauer and C. M. Agrawal, Biomaterials, 17, 93 (1996)). Sustained release requires the injection of 10-100 μm PLGA particles with a pharmacologically active substance loaded therein, but this gives rise to a pain or inflammation.

As an alternative solution to the problems, International Patent Publication No. WO 2005/117830 describes a pre-formulation comprising at least one neutral diacyl lipid and/or tocopherol, at least one phospholipid, and at least one biocompatible, oxygen containing, low viscosity organic solvent. Another alternative is described in the International Patent Publication No. WO 2006/075124 which concerns a pre-formation comprising at least one diacyl glycerol, at least one phosphatidyl choline, at least one oxygen containing organic solvent, and at least one somatostatin analogue. These preformulations allow the sustained release of a pharmacologically active substance in vivo for two or more weeks, and do not form lactic acid or glycolic acid degradations from their polymer systems, thus not causing pain or inflammation. However, there is a problem with the formulations in that their necessarily organic solvent incurs a reduction in the activity of some pharmacologically active substances (H. Ljusberg-Wahre, F. S. Nielse, 298, 328-332 (2005); H. Sah, Y. Bahl, Journal of Controlled Release 106, 51-61(2005)). In addition, nowhere is a composition capable of reinforcing sustained release other than provided by the liquid crystalline phase mentioned in the patent publications.

Culminating in the present invention, intensive and thorough research of the present inventors into the sustained release formulation led to the findings that a lipid pre-concentrate comprising a) at least one liquid crystal former; b) at least one neutral phospholipid; c) at least one liquid crystal hardener; d) at least one anionic anchoring agent, which exists as a lipid liquid phase in the absence of aqueous fluid and shifts into a liquid crystal upon exposure to aqueous fluid; and e) a pharmaceutical composition comprising at least one cationic pharmacologically active substance with a net charge of (+) in addition to the pre-concentrate shows enhanced sustained release profile as a result of the ionic interaction between cationic pharmacologically active substance and anionic anchoring agent.

A description is given of the prior arts relevant to the present invention, infra.

International Patent Publication No. WO 2009/024795 describes a composition comprising a low viscosity mixture of at least one non-polymeric controlled-release matrix, at least one biocompatible organic solvent, at least one peptide pharmacologically active substance; and at least one fat-soluble acid. However, the indispensable organic solvent reduces the activity of some pharmacologically active substances, and the fat-soluble acid does not serve as a typical acid, but aims only to act as a dissolution stabilizer, which is different from the present invention in that the composition is irrelevant to the enhancement of the sustained release of pharmacologically active substances.

International Patent Publication No. WO 2008/152401 describes a composition for the delayed delivery of a peptide pharmacologically active substance comprising at least one positively charged peptide ion and at least one salt of pharmacologically active substance that includes negatively charged counter ion, and a controlled-release delivery vehicle. In this composition, however, the controlled-release delivery vehicle may include a bio-resistant polymer such as PLGA, while the pharmacologically active substance is limited to peptides. In addition, the salt of the pharmacologically active substance comprising the counter ion, a kind of pharmacologically active substance applied to controlled-release matrixes, is different from that of the present invention because of no interactions between the controlled-release matrix and the ion.

In Japanese Patent Publication. No. 62-1239226, a pharmacologically active substance containing a cationic group which is dissolved or dispersed in a solution is described to occur ion exchange with the carboxylic acid group of hyaluronic acid, thereby inducing the slow diffusion of a pharmacologically active substance. However, it is difficult for the hyaluronic acid sustained release formulation to allow for sufficient sustained release in degradation even if full consideration of the ion exchange between hyaluronic acid and the cationic pharmacologically active substance associated with the viscous hyaluronic acid. In addition, considering that the sustained release mechanism is based on the viscosity of hyaluronic acid, it is more difficult for a pharmacologically active substance with higher water solubility to achieve a desired sustained release. That is why the reference is different from the present invention.

International Patent Publication No. WO 1999/033491 describes sustained release pharmaceutical compositions of an ionic pharmacologically active substance containing ionic compounds which are charged oppositely to the ionic pharmacologically active substance and which are capable of elevating the hydrophobicity of the substance. The composition is similar to that of the present invention in that the sustained release is enhanced using an ionic compound having a counter ion, and that it can take a subcutaneous injection. However, it is different from the present invention as follows. The sustained release of pharmacologically active substance is not based on direct ionic interaction between the pharmacologically active substance and the counter ion compound, but relies on an improvement in the hydrophobicity of the pharmacologically active substance. In addition, this composition was found to appear temporal sustained release only within several hours, as measured in rats that was injected subcutaneously to the back region.

U.S. Pat. No. 7,731,947 describes a composition comprising: a particle formulation comprising an interferon, sucrose, methionine, and a citrate buffer suspends organic solvent such as benzyl benzoate. In one Example, it is described that phosphatidyl choline is dissolved together with vitamin E (tocopherol) in an organic solvent and is used to disperse the particle formulation therein. However, this composition is different from the present invention in that the composition is used to disperse solid particles and does not allow the formation of liquid crystals.

U.S. Pat. No. 7,871,642 describes a method of preparing a dispersion for delivering a pharmacologically active substance, comprising dispersing a homogeneous mixture of a phospholipid, a polyoxyethylene coemulsifier, triglyceride and ethanol in water, wherein polyoxyethylene sorbitan fatty acid ester (polysorbate) or polyoxyethylene vitamin E derivatives can be used for one of polyoxyethylene surfactants. However, polyoxyethylene sorbitan fatty acid esters and polyoxyethylene vitamin E derivatives, derived by conjugating the hydrophilic polymer polyoxyethylene to sorbitan fatty acid ester and vitamin E, respectively, are quite different in structure from sorbitan fatty acid ester and vitamin E. They are usually used as hydrophilic surfactants utilizing the property of polyoxyethylene, which is different from the component of the present invention.

U.S. Pat. No. 5,888,533 describes a fluid composition for forming a solid implant, comprising: a non-polymeric, water-insoluble, biodegradable material; and a biocompatible, organic solvent that solubilizes the material to a minimum and is miscible or dispersible in water or body fluids, and capable of diffusing-out or leaching from the composition into body fluid upon placement within a body, whereupon the non-polymeric, water-insoluble, biodegradable material coagulates or precipitates to form the solid implant. In this composition, sterols, cholesteryl esters, fatty acids, fatty acid glycerides, sucrose fatty acid esters, sorbitan fatty acid esters, fatty alcohols, the combination of fatty alcohols with fatty acids through esterification, anhydrides of fatty acids, phospholipids, lanolin, lanolin alcohols, and combinations thereof are described as the non-polymeric material, and ethanol is used as one of solvent. However, differences from the present invention reside in that this composition cannot form liquid crystals and is designed to form solid implants by simple coagulation or precipitation of water-insoluble materials and that a lot of the organic solvent is necessarily used.

### DISCLOSURE OF INVENTION

### Technical Problem

It is therefore an object of the present invention to provide a sustained-release lipid pre-concentrate for enhancing the sustained release of cationic pharmacologically active substances, comprising an anionic anchoring agent.

It is another object of the present invention to provide a sustained-release lipid pre-concentrate comprising an anionic anchoring agent without problems of safety and biodegradability.

### Solution to Problem

In accordance with an aspect thereof, the present invention addresses a sustained-release lipid pre-concentrate comprising: a) at least one liquid crystal former; b) at least one neutral phospholipid; c) at least one liquid crystal hardener; and d) at least one anionic anchoring agent, which exists as a lipid liquid phase in the absence of aqueous fluid and forms into a liquid crystal upon exposure to aqueous fluid.

In accordance with another aspect thereof, the present invention addresses a pharmaceutical composition comprising the sustained-release lipid pre-concentrate and e) at least one cationic pharmacologically active substance with a net charge of (+), in which the cationic pharmacologically active substance exhibits enhanced sustained release as a result of ionic interaction between the anionic anchoring agent and the cationic pharmacologically active substance.

Below, a detailed description will be given of each component.

a) Liquid Crystal Former

The liquid crystal former used in the present invention is responsible for the formation of non-lamellar liquid crystals, and may be selected from sorbitan unsaturated fatty acid ester, monoacyl glycerol, diacyl glycerol, and a combination thereof.

For use as a liquid crystal former in the present invention, the sorbitan unsaturated fatty acid ester preferably has two or more —OH (hydroxyl) groups in the polar head. This sorbitan unsaturated fatty acid ester may be represented by the following Chemical Formula 1. The compound of Chemical Formula 1 is sorbitan monoester where R¹═R²═OH, R³═R, and sorbitan diester where R¹═OH, R²═R³═R, R being an alkyl ester group of 4 to 30 carbon atoms with at least one unsaturated bond.

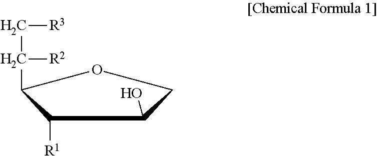

In detail, the sorbitan unsaturated fatty acid ester of the present invention may be selected from sorbitan monoester, sorbitan sesquiester, sorbitan diester and a combination thereof, which can be derived from fatty acids that can be obtained from whale oils and fish oils as well as vegetable oils (e.g., coconut oil, castor oil, olive oil, peanut oil, rapeseed oil, corn oil, sesame oil, cotton seed oil, soybean oil, sunflower seed oil, safflower oil, linseed oil, etc.), and animal fats and oils (e.g., milk fat, lard, and beef tallow).

Sorbitan monoester is a compound in which one fatty acid group is attached to sorbitan via an ester bond, and may be selected from among sorbitan monooleate, sorbitan monolinoleate, sorbitan monopalmitoleate, sorbitan monomyristoleate, and a combination thereof.

Sorbitan sesquiester is a compound in which 1.5 fatty acid groups, on average, are attached to sorbitan via an ester bond, and may be selected from among sorbitan sesquioleate, sorbitan sesquilinoleate, sorbitan sesquipalmitoleate, sorbitan sesquimyristoleate, and a combination thereof.

Sorbitan diester is a compound in which two fatty acid groups are attached to sorbitan via an ester bond, and be able to select more than one from among sorbitan dioleate, sorbitan dilinoleate, sorbitan dipalmitoleate, sorbitan dimyristoleate, and a combination thereof.

For use in the present invention, sorbitan unsaturated fatty acid ester is preferably selected from sorbitan monooleate, sorbitan monolinoleate, sorbitan monopalmitoleate, sorbitan monomyristoleate, sorbitan sesquioleate, and a combination thereof.

Monoacyl glycrol, which can be used as another liquid crystal former in the present invention, consists of glycerine as the polar head and one fatty acid as a tail with linkage of ester bond, while diacyl glycerol contains the polar head of glycerine that be linked with two fatty acid via ester bonds. In the present invention, fatty acid group which can be linked with mono- or diacyl glycerol may contain the same or different numbers of carbon atoms ranging from 4 to 30, and may independently be saturated or unsaturated. The fatty acid may be able to select more than one from among the groups consisting of palmitic acid, palmitoleic acid, lauric acid, butyric acid, valeric acid, caproic acid, enanthic acid, caprylic acid, pelargonic acid, capric acid, myristic acid, myristoleic acid, stearic acid, arachidic acid, behenic acid, lignoceric acid, cerotic acid, linolenic acid, alpha-linolenic acid (ALA), eicosapentaenoic acid (EPA), docosahexaenoic acid (DHA), linoleic acid (LA), gamma-linoleic acid (GLA), dihomo gamma-linoleic acid (DGLA), arachidonic acid (AA), oleic acid, vaccenic acid, elaidic acid, eicosanoic acid, erucic acid, nervonic acid, and a combination thereof.

Concretely, the monoacyl glycerol of the present invention, is able to select more than one from among the present invention include glycerol monobutyrate, glycerol monobehenate, glycerol monocaprylate, glycerol monolaurate, glycerol monomethacrylate, glycerol monopalmitate, glycerol monostearate, glycerol monooleate, glycerol monolinoleate, glycerol monoarchidate, glycerol monoarchidonate, glycerol monoerucate, and a combination thereof. Preferably, the glycerol monooleate (GMO) of the following chemical formula 2 can be used.

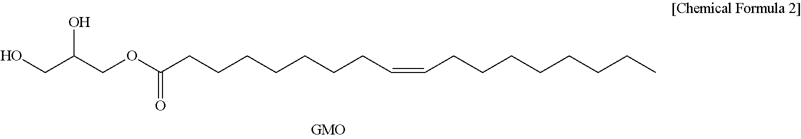 [SMILES: CCCCCCCC/C=C\CCCCCCCC(=O)OCC(O)CO]

The diacylglycerol of the present invention, is able to select more than one from among glycerol dibehenate, glycol dilaurate, glycerol dimethacrylate, glycerol dipalmitate, glycerol distearate, glycerol dioleate, glycerol dilinoleate, glycerol dierucate, glycerol dimyristate, glycerol diricinoleate, glycerol dipalmitoleate, and a combination thereof. Preferably, the glycerol dioleate (GDO) of the following chemical formula 3 can be used.

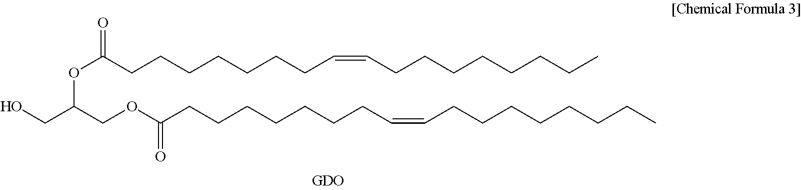 [SMILES: CCCCCCCC/C=C\CCCCCCCC(=O)OCC(CO)OC(=O)CCCCCCC/C=C\CCCCCCCC]

b) Neutral Phospholipid

Phospholipids of present invention are essential for the construction of lamellar structures, such as liposomes, in conventional techniques, but, cannot form a non-lamellar phase structure, such as a liquid crystal, by themselves. However, phospholipids can participate in the liquid crystal former-driven formation of non-lamellar phase structures, serving to stabilize the resulting liquid crystals.

The phospholipid useful in the present invention is preferably neutral, and contains a saturated or unsaturated alkyl ester group of 4 to 30 carbon atoms with a polar head. The phospholipid may be selected more than one from among phosphatidylcholine, phosphatidylethanolamine, phosphatidylinositol, sphingomyelin, and a combination thereof according to the structure of the polar head.

Phospholipids are found in plants and animals such as soybeans and eggs. In phospholipids, long alkyl ester groups which account for the hydrophobic tails include saturated fatty acid chains such as mono- and dipalmitoyl, mono- and dimyristoyl, mono- and dilauryl, and mono- and distearyl, and unsaturated fatty acid chains such as mono- or dilinoleyl, mono- and dioleyl, mono- and dipalmitoleyl, and mono- and dimyristoleyl. In addition, the saturated and unsaturated fatty acid esters may coexist in phospholipids.

c) Liquid Crystal Hardener

The liquid crystal hardener of the present invention cannot form a non-lamellar structure, unlike the liquid crystal former, nor a lamellar structure such as liposome) unlike phospholipids, by itself. However, the liquid crystal hardener contributes to the liquid crystal former-driven formation of non-lamellar phase structures by increasing the curvature of the non-lamellar structures to enhance the ordered co-existence of oil and water. In the interests of this function, the liquid crystal hardener is advantageously required to have a highly limited polar moiety and a bulky non-polar moiety inside its molecular structure.

In practice, however, biocompatible substance which are injectable into the body can be selected as the liquid crystal hardener of the present invention only via direct and repeated experiments, especially. As a result, liquid crystal hardeners suitable for the composition of the present invention has molecular structures which are different from one another and thus cannot be elucidated as having only one molecular structure. The common structural feature deduced by observation of all of the liquid crystal hardeners identified is that they are free of ionizable groups, such as carboxyl and amine groups, and have hydrophobic moieties comprising a bulky triacyl group with 15 to 40 carbon atoms or carbon ring structure. Preferred examples of the liquid crystal hardener of the present invention may be free of ionizable groups, such as carboxyl and amine groups, and have at most one hydroxyl and ester group as a weak polar head, with hydrophobic moieties including a bulky triacyl group with 20 to 40 carbon atoms or carbon ring structure. Examples of the liquid crystal hardener of the present invention may include, but are not limited to, triglyceride, retinyl palmitate, tocopherol acetate, cholesterol, benzyl benzoate, ubiquinone, and a combination thereof. Preferably, the liquid crystal hardener can be selected more than one from among tocopherol acetate, cholesterol, and a combination thereof.

d) Anionic Anchoring Agent

The term “ionic anchoring agent,” as used herein, is intended to encompass all agents that allow for the sustained release of a pharmacologically active substance through ionic interaction between the pharmacologically active substance and the anchoring agent with the oppositely charged ion with the etymological meaning of holding fast by or as if by an anchor.

In the present invention, the anionic anchoring agent of the sustained release lipid pre-concentrate is used to enhance the sustained release of a cationic pharmacologically active substance.

For use in the present invention, the anionic anchoring agent may be a compound comprising a polar head group which can at least be selected more than one from among carboxylate, phosphate, sulfate, sulfonate, and a combination thereof, with a hydrophobic tail of 4 to 40 carbon atoms.

Concretely, examples of the anionic anchoring agent containing the polar head of carboxylate include, but are not limited to, palmitic acid, palmitoleic acid, lauric acid, butyric acid, valeric acid, caproic acid, enanthic acid, caprylic acid, pelargonic acid, capric acid, myristic acid, myristoleic acid, stearic acid, arachidic acid, behenic acid, lignoceric acid, cerotic acid, linolenic acid, alpha-linolenic acid (ALA), eicosapentaenoic acid (EPA), docosahexaenoic acid (DHA), linoleic acid (LA), gamma-linoleic acid (GLA), dihomo gamma-linoleic acid (DGLA), arachidonic acid (AA), oleic acid, vaccenic acid, elaidic acid, eicosanoic acid, erucic acid, nervonic acid, benzoic acid, sorbic acid, pamoic acid, lipoic acid, and a combination thereof.

The anionic anchoring agent with the phosphate polar head may be selected from among phosphatidylserine, phosphatidylglycerine, phosphatidylinositol, phosphatidic acid, and a combination thereof, with 1,2-dipalmitoyl-sn-glycero-3-phosphate, preferably.

The anionic anchoring agent with the sulfate polar head can be selected more than one from among, but not limited to, lauryl sulfate, cholesteryl sulfate, and a combination thereof.

The anionic anchoring agent with the sulfonate polar head can be selected more than one from among, but not limited to, benzene sulfonate, dodecylbenzene sulfonate, and a combination thereof.

e) Cationic Pharmacologically Active Substance

The term of cationic pharmacologically active substance in present invention refers to a pharmacologically active ingredient positively charged or with a net charge of (+).

The cationic pharmacologically active substance may be in the form of at least one selected from among a primary amine, a secondary amine, a tertiary amine, an aromatic amine, a sulfonium, an iodonium, an ammonium, a phosphonium, a pyridinium, a thiazolinium, an imidazolinium, a sulfoxonium, an isothiouronium, an azetidinium, and a diazonium.

Concretely, examples of the cationic pharmacologically active substance useful in the present invention include leuprolide, triptorelin, goserelin, nafarelin, buserelin, histrelin, deslorelin, meterelin, gonadrelin, entecavir, anastrozole, rivastigmin, acapodene, abiraterone, tibolone, fentanyl, tacrolimus, methotrexate, tamsulosin, dutasteride, finasteride, solifenacin, tadalafil, donepezil, olanzapine, risperidone, aripiprazole, naltrexone, varenicline, ropinirole, latanoprost, olopatadine, progesterone, ketotifen, montelukast, human growth hormone, tramadol, diazepam, diclofenac, pilocarpine, levocabastine, timolol, betaxolol, carteolol, levobunolol, epinephrine, dipivefrine, clonidine, apraclonidine, indomethacin, acyclovir, testosterone, statin, nifedipine, voriconazole, clotrimazole, ketoconazole, fulvestrant, fibrate, octreotide, estradiol, cortisone, amphotericin B, chlorhexidine, corticosteroid, cyclosporine A, desmopressin, somatostatin, calcitonin, oxytocin, vasopressin, follitropin-alpha or beta, thyrotropin alpha, secretin, bradykinin, hypotensive tissue hormone, insulin or insulin derivatives, interferon, a tuftsin, magainin, indolicidin, protegrin, polymyxin, gramicidin, vapreotide, exenatide, liraglutide, CJC-1131, AVE010, LY548806 and TH-0318, BIM 51077, degarelix, glucagon, defensin, histatin, galantamine, memantine, tacrine, alprazolam, tandospirone citrate, diphenylpyraline, cimetidine, isothipendyl, phenylephrin, procainamide, quinidine, isosorbide, nicorandil, amlodipine, atenolol, pherospin, floxacin, cephalexin, cefcapene pivoxil, sulfamethoxazole, tetracycline, metronidazole, indapamide, papaverine, bromhexine, ticlopidine, carbetapentane, phenylpropanolamine, cetirizine, mycin, acetaminophen, coxib, morphine, codeine, oxycodone, buprenorpine, prazosin, hydroxyzine, buspirone, chlordiazepoxide, meprobamate, trifluoperazine, clorazepate, abciximab, eptifibatide, tirofiban, lamifiban, clopidogrel, dicoumarol, heparin, warfarin, phenobarbital, clobazam, felbamate, carbamazepine, oxcarbazepine, vigabatrin, progabide, tiagabine, topiramate, gabapentin, pregabalin, ethotoin, paramethadione, beclamide, primidone, levetiracetam, acetazolamide, lamotrigine, phenacemide, valpromide, valnoctamide, repaglinide, metformin, glitazone, miglitol, vildagliptin, sitagliptin, tolbutamide, acetohexamide, tolazamide, glyburide, glimepiride, gliclazide, glipizide, chlorpropamide, pseudoephedrine, oxymetazoline, mepyramine, antazoline, diphenylhyramine, carbinoxamine, doxylamine, clemastine, dimenhydrinate, peniramin, triprolidine, chlorcyclizine, meclizine, promethazine, trimeprazine, cyproheptadine, azatadine, dextromethorphan, noscapine, chlorambucil, lomustine, betamethasone, aspirin, piroxicam, carprofen, flurbiprofen, timolol, nadolol, theobromine, doxorubicin, alprenolol, sotalol, acebutolol, atenolol, bisoprolol, esmolol, metoprolol, nebilet, carvedilol, celiprolol, labetalol, adalimumab, azathioprine, chloroquine, Dphenicillamine, etanercept, aurothio, auranofin, infliximab, leflunomide, sulfasalazine, prednisone, triamcinolone, aldosterone, benorilate, diflunisal, acemetacin, bromfenac, etodolac, nabumetone, sulindac, tolmetin, ketololac, mefenamic acid, phenylbutazone, metamizole, oxyphenbutazone, sulfinpyrazone, meloxicam, nimesulide, ziprasidone, fluspirilene, penfluridol, ampakine, dexedrin, fenfluramine, phentermine, orlistat, acarbose, rimonabant, sildenafil, carbenicillin indanyl, bacampicillin, ampicillin, penicillin G, nelfinavir, virazole, benzalkonium, griseofulvin, thiabendazole, oxfendazole, oxibendazole, morantel, co-trimoxazole, alfaxalone, etomidate, levodopa, bromocriptine, pramipexole, pergolide, selegiline, trihexyphenidyl, benztropine, phencyclidine, orphenadrine, amantadine, galantamine, rifampin, cefazolin, imipenem, aztreonam, sulfamethoxazole, trimethoprim, teicoplanin, mupirocin, nalidixic acid, sulbactam, clavulanic acid, nystatin, isocarboxazid, phenelzine, tranylcypromine, zidovudine, dideoxyinosine, zalcitabine, nevirapine, lamivudine, saquinavir, delavirdine, methylphenidate, cabergoline, ondansetron, domperidone, peridol, chloropromazine, prochlorperazine, metoclopramide, alizaprid, loperamide, cisapride, thioridazine, amitriptyline, bupropion, chlordiazepoxide, citalopram, clozapine, fluoxetine, fluphenazine, fluvoxamine, hydrazine, lorazepam, loxapine, mirtazapine, molindone, nefasodone, nortriptyline, paroxetine, quetiapine, sertraline, thiothixene, trazodone, venlafaxine, fentanyl, methadone, oxymorphone, valporate, pentoin, albuterol, baclofen, carisoprodol, chlorzoxazone, cyclobenzaprine, dantrolene, metaxalone, orphenadrine, pancuronium, dicyclomine, a combination thereof, and a pharmaceutically acceptable salt thereof, but are not limited thereto.

Preferably, the cationic pharmacologically active substance may be selected from the groups consisting of leuprolide, triptorelin, goserelin, nafarelin, buserelin, histrelin, deslorelin, meterelin, gonadrelin, entecavir, anastrozole, rivastigmin, acapodene, abiraterone, tibolone, fentanyl, tacrolimus, methotrexate, tamsulosin, dutasteride, finasteride, solifenacin, tadalafil, donepezil, olanzapine, risperidone, aripiprazole, naltrexone, varenicline, ropinirole, latanoprost, olopatadine, progesterone, ketotifen, montelukast, and a combination thereof. More preferably, selection may be made of the cationic pharmacologically active substance from a group consisting of leuprolide, triptorelin, goserelin, nafarelin, buserelin, histrelin, deslorelin, meterelin, gonadrelin, entecavir, anastrozole, rivastigmin, a combination thereof, and a pharmaceutically acceptable salt thereof.

In the composition of pre-concentrate of suitable liquid crystal of the present invention, the weight ratio between components of a) and b) is in a range of from 10:1 to 1:10, and preferably in a range of 5:1 to 1:5. The weight ratio of a)+b) to c) falls within the range of from 1,000:1 to 1:1 and preferably in a range of 100:1 to 1:1, and more preferably within the range of from 50:1 to 2:1. The suitable weight ratio of a)+b)+c) to d), it ranges from 5,000:1 to 5:1, and preferably from 500:1 to 10:1. Given these weight ranges, the components efficiently guarantee the sustained release attributable to liquid crystals and the anionic anchoring agent-induced improvement in sustained release.

Generally, the pharmaceutical composition of the present invention may comprise a suitable weight ratio of a)+b)+c)+d) to e) in the range of from 10,000:1 to 2:1, which may vary depending on the kind of the pharmacologically active substance, the kind of formulation to be applied, desired release patterns, and the content of the pharmacologically active substance required in the medical field.

As used herein, the term “aqueous fluid” is intended to include water and body fluid such as a mucosal solution, a tear, a sweat, a saliva, gastrointestinal fluid, an extravascular fluid, an extracellular fluid, an interstitial fluid, and a blood plasma. When brought into body surfaces, regions or cavities (e.g. inside the body) whose external environments are formed for by aqueous fluids, the pharmaceutical composition of the present invention undergoes transition from a liquid phase to a liquid crystalline phase with a semi-solid appearance. That is, the pharmaceutical composition of the present invention is a pre-concentrate which exists as a liquid state before application to the human body and shifts into a liquid crystalline phase sustained release behavior within the body.

The liquid crystals formed by the pharmaceutical composition of the present invention have a non-lamellar phase structure in which oil and water are in an ordered mixture and arrangement without distinction between inner and out phases. The ordered arrangement of oil and water renders the non-lamellar phase structure of a mesophase, which is a state of between liquid and solid. The pre-concentrate of the present invention is different from conventional compositions that are lamellar structures, such as micelles, emulsions, microemulsions, liposomes, and lipid bilayers, which have been widely used in designing pharmaceutical formulations. Such lamellar structures are in oil in water (o/w) or water in oil (w/o) type in which there have an arrangement with inner and out phases.

The term “liquid crystallization,” as used herein, refers to the formation of liquid crystals having a non-lamellar phase structure from the pre-concentrate upon exposure to aqueous fluid.

The sustained release lipid pre-concentrate of the present invention may be prepared at room temperature from a) at least one liquid crystal former, b) at least one neutral phospholipid, c) at least one liquid crystal hardener, and d) at least one anionic anchoring agent, and if necessary, by heating or using a homogenizer. The homogenizer may be a high-pressure homogenizer, an ultrasonic homogenizer, a bead mill homogenizer, and etc.

As described above, the sustained-release lipid pre-concentrate of the present invention may be a pharmaceutical composition which exists in a liquid phase in the absence of aqueous fluid and forms into liquid crystals in the presence of aqueous fluid. As it turns to a pharmaceutical composition which can be applied to the body using a method selected from among injection, coating, dripping, padding, oral administration, and spraying, the pre-concentrate of the present invention may be preferably formulated into various dosage forms including injections, ointments, gels, lotions, capsules, tablets, solutions, suspensions, sprays, inhalants, eye drops, adhesives, plaster and pressure sensitive adhesives, and more preferably into injections.

Particularly, when an injection route is taken, the pre-concentrate of the present invention may be administered by subcutaneous or intramuscular injection or other injection routes depending on the properties of the pharmacologically active substance used.

The pharmaceutical composition of the present invention may be preferably in the formulation form selected from among injections, ointments, gels, lotions, capsules, tablets, solutions, suspensions, sprays, inhalants, eye drops, adhesives, plaster and pressure sensitive adhesives, and more preferably into injections.

The pharmaceutical composition of the present invention may be prepared by adding a pharmacologically active substance to the pre-concentrate of the present invention. As needed, heat or a homogenizer may be used in the preparation of the pharmaceutical composition of the present invention, but this is not a limiting factor to the present invention.

The dose of the pharmaceutical composition of the present invention adheres to the well-known dose of the pharmacologically active substance employed, and may vary depending on various factors including the patient's condition, age and sex. It may be administered orally or parenterally depending on the properties of the pharmacologically active substance.

In accordance with a further aspect thereof, the present invention contemplates a method of maintaining pharmaceutical efficacy through the sustained release of a pharmacologically active substance by administering the pharmaceutical composition of the present invention to a mammal including a human, and the use of the pharmaceutical composition for the sustained release of a pharmacologically active substance.

### Advantageous Effects of Invention

As described hitherto, the sustained-release lipid pre-concentrate and the pharmaceutical composition according to the present invention, guarantee excellent sustained release of the pharmacologically active substance on the basis of ionic interaction between the anionic anchoring agent and the cationic pharmacologically active substance within the liquid crystals formed.

### BRIEF DESCRIPTION OF DRAWINGS

FIG. 1 is a schematic view illustrating partial or entire ionic interaction between anionic anchoring agents and cationic pharmacologically active substances within the sustained-release lipid pre-concentrate.

FIG. 2 shows in vivo biodegradability of the compositions of Examples 2 and 13 and Comparative Examples 1 to 7.

FIG. 3 shows in vivo drug release behaviors of the pharmacologically active substances (leuprolide) of the compositions of Example 21 and Comparative Examples 15 and 21.

FIG. 4 shows in vivo drug release behaviors of the pharmacologically active substances (entecavir) of the compositions of Example 24 and Comparative Examples 18 and 22.

FIG. 5 shows phase change behaviors of the compositions of Example 13 and Comparative Example 1 upon exposure to aqueous fluid.

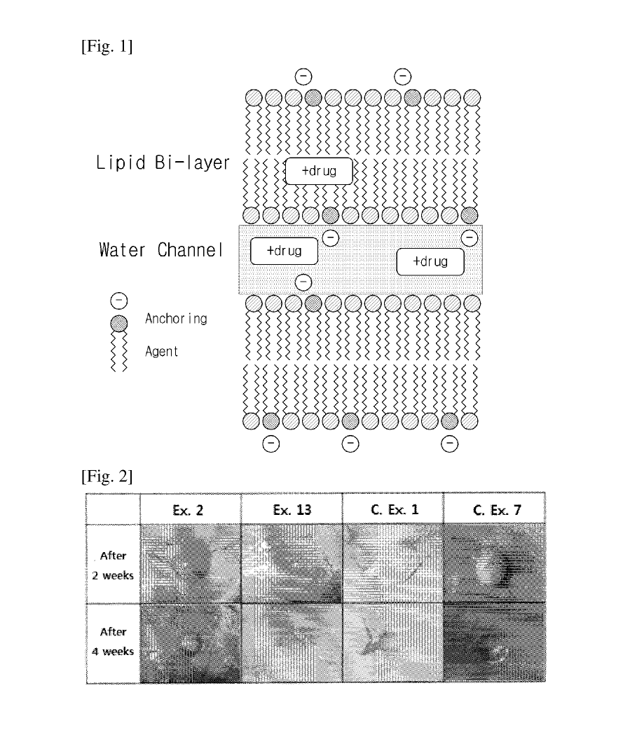

### MODE FOR THE INVENTION

The following non-limiting Examples serve to illustrate selected embodiments of the invention. It will be appreciated that variations in proportions and alternatives in elements of the components shown will be apparent to those skilled in the art and are within the scope of embodiments of the present invention.

The additives and excipients used in the present invention satisfied the requirements of the pharmacopoeia and were purchased from Aldrich, Lipoid, Croda, and Seppic.

### Examples 1 to 19

### Preparation of Lipid Pre-Concentrates

At the weight ratios given in Table 1, below, liquid crystal formers, neutral phospholipids, liquid crystal hardeners, and anionic anchoring agents were added optionally in a solvent.

In Examples 1 to 19, the substances were mixed in a water bath maintained at 20-75° C. using a homogenizer (PowerGen model 125, Fisher) for 0.5-3 hrs at 1000-3000 rpm. Then, the resulting lipid solutions were left at room temperature to come to thermal equilibrium at 25° C. before being loaded into 1 cc disposable syringes. The lipid solutions were injected into water (2 g of deionized water) to prepare pre-concentrates of the present invention.

TABLE 1

| | Example | | | | | | | | | | | | | | | | | | |
|---|---|---|---|---|---|---|---|---|---|---|---|---|---|---|---|---|---|---|---|
| (Unit: mg) | 1 | 2 | 3 | 4 | 5 | 6 | 7 | 8 | 9 | 10 | 11 | 12 | 13 | 14 | 15 | 16 | 17 | 18 | 19 |
| Benzoic acid | 12.5 | 17.5 | | | | | | | | | | | | | | | | | |
| Sorbic acid | | | 3.5 | 21.5 | | | | | | | | | | | | 13.5 | | | |
| Stearic acid | | | | | | | | | | | | | | | | | 7 | 1 | |
| Phosphatidic | | | | | 0.1 | 0.5 | 1 | 8.5 | 20 | | | | | | | | | | |
| acid | | | | | | | | | | | | | | | | | | | |
| Lauryl | | | | | | | | | | 0.1 | 0.9 | 1.8 | 2.5 | | | | | | |
| sulfate | | | | | | | | | | | | | | | | | | | |
| Dodesyl | | | | | | | | | | | | | | 0.2 | 1.6 | | | | 5 |
| Benzene | | | | | | | | | | | | | | | | | | | |
| sulfonate | | | | | | | | | | | | | | | | | | | |
| Sorbitan | 43.4 | | 60.1 | 55.0 | 40.0 | | 40.0 | 32.8 | | 60.1 | | | 35.9 | 45.1 | | | | | |
| monooleate | | | | | | | | | | | | | | | | | | | |
| Sorbitan | | 42.4 | | | | 50.3 | | | 43.8 | | 46.4 | 45.8 | | | 45.7 | | | | |
| sesquioleate | | | | | | | | | | | | | | | | | | | |
| Glycerol | | | | | | | | | | | | | | | | 46.2 | 51.6 | | |
| monooleate | | | | | | | | | | | | | | | | | | | |
| Glycerol | | | | | | | | | | | | | | | | | | 52.0 | |
| dioleate | | | | | | | | | | | | | | | | | | | |
| Phytanyl | | | | | | | | | | | | | | | | | | | 63.2 |
| glycerate | | | | | | | | | | | | | | | | | | | |
| Phosphatidyl | 36.6 | 50.9 | 35.2 | | 50.1 | | 48.0 | 43.7 | | | 53.5 | | 46.2 | 45.1 | | 33.5 | 37.6 | 30.3 | |
| choline | | | | | | | | | | | | | | | | | | | |
| Phosphatidyl | | | | 35.1 | | 45.2 | | | 50.0 | 30.0 | | 40.7 | | | 35.6 | | | | 26.3 |
| ethanolamine | | | | | | | | | | | | | | | | | | | |
| Tocophorol | 14.3 | | 4.7 | 9.9 | 9.9 | | | | 6.3 | | | | 12.8 | 9.8 | 10.6 | 14.5 | | 7.6 | |
| acetate | | | | | | | | | | | | | | | | | | | |
| Cholesterol | | 6.7 | | | | 4.5 | | 18.0 | | 9.9 | | 13.4 | | | 8.1 | | | | 3.2 |
| Benzyl | | | | | | | 7.0 | | | | | | | | | | 10.8 | | |
| benzoate | | | | | | | | | | | | | | | | | | | |
| Ubiquinone | | | | | | | | | | | 0.1 | | | | | | | | |
| Ethanol | 5.7 | | | | | | 5 | 5.5 | | | | | 5.1 | | | 5.8 | | 10.1 | 7.4 |

| | |
|---|---|
| Form in | Liquid Crystal |
| aqueous | |
| phase | |

### Examples 20 to 30

### Preparation of Pharmaceutical Compositions

Liquid crystal formers, neutral phospholipids, liquid crystal hardeners, anionic anchoring agents, and cationic pharmacologically active substances were mixed at the weight ratios given in Table 2, below, optionally in solvents.

In Examples 20 to 30, the substances were homogeneously mixed in a water bath maintained at 20-75° C. using a homogenizer (PowerGen model 125. Fisher) for 0.5-3 hrs at 1000-3000 rpm. The resulting lipid solutions were left at room temperature to come to thermal equilibrium at 25° C., followed by adding each of the pharmacologically active substances leuprolide, entecavir, risperidone, anastrozole, interferon, and exenatide thereto. Then, the substances were homogenized using a homogenizer at 1000-3000 rpm for about 5-30 mins to prepare pharmaceutical compositions in a solution phase.

TABLE 2

| | Example | | | | | | | | | | |
|---|---|---|---|---|---|---|---|---|---|---|---|
| (Unit: mg) | 20 | 21 | 22 | 23 | 24 | 25 | 26 | 27 | 28 | 29 | 30 |
| Leuprolide | 3.75 | 3.75 | 3.75 | | | | | | | | |
| Entecavir | | | | 2.3 | 2.3 | | | | | | |
| Risperidone | | | | | | 10 | 10 | | | | |
| Anastrozole | | | | | | | | 5 | 5 | | |
| Interferon | | | | | | | | | | 0.042 | |
| Exenatide | | | | | | | | | | | 1 |
| Benzoic acid | 12.5 | | | | | | | | | | |
| Sorbic acid | | | | | | 21.5 | | 3.5 | | 7 | |
| Stearic acid | | | | | | | | | | | 12.9 |
| Phosphatidic acid | | 1 | | 8.5 | | | 0.1 | | 3 | | |
| Lauryl sulfate | | | 1.8 | | 0.9 | | | | | | |
| Sorbitan | 43.4 | 35 | | 32.8 | | 51.0 | 40.0 | 36.3 | 32.6 | | |
| monooleate | | | | | | | | | | | |
| Sorbitan | | | 45.8 | | 35.0 | | | | | | |
| sesquioleate | | | | | | | | | | | |
| Glycerin | | | | | | | | | | 51.6 | |
| monooleate | | | | | | | | | | | |
| Glycerin | | | | | | | | | | | 54.0 |
| dioleate | | | | | | | | | | | |
| Phosphatidyl | 36.6 | 45 | | 43.7 | 55.0 | | 50.1 | 46.6 | 54.9 | | 40.2 |
| choline | | | | | | | | | | | |
| Phosphatidyl | | | 40.7 | | | 38.2 | | | | 37.6 | |
| ethanolamine | | | | | | | | | | | |
| Tocopherol acetate | 14.3 | 15 | | 12.6 | 10.0 | 10.8 | 9.9 | 11.9 | 8.2 | | |
| Cholesterol | | | 13.4 | 5.5 | | | | | 4.3 | | |
| Benzyl | | | | | | | | | | 10.8 | |
| benzoate | | | | | | | | | | | |
| Ubiquinone | | | | | | | | | | | 0.1 |
| Ethanol | 5.7 | 5 | | 5.5 | | | | 5.2 | | | 5.7 |

| | |
|---|---|
| Form in aqueous | Liquid Crystal |
| phase | |

### Comparative Examples 1 to 13

### Preparation of Pre-Concentrates Devoid of Anionic Anchoring Agent

At the weight ratios given in Table 3, below, liquid crystal formers, neutral phospholipids, and liquid crystal hardeners were mixed, optionally in a solvent.

In Comparative Examples 1 to 13, the substances were mixed in a water bath maintained at 25-75° C. using a homogenizer (PowerGen model 125, Fisher) for 10 mins at 3000 rpm. Then, the resulting lipid solutions were left at room temperature to come to thermal equilibrium at 25° C. before being loaded into 1 cc disposable syringes. The lipid solutions were injected into water (2 g of deionized water) to prepare pre-concentrates according to Comparative Examples 1 to 13.

TABLE 3

| | Comparative Example | | | | | | | | | | | | |
|---|---|---|---|---|---|---|---|---|---|---|---|---|---|
| (Unit: mg) | 1 | 2 | 3 | 4 | 5 | 6 | 7 | 8 | 9 | 10 | 11 | 12 | 13 |
| Sorbitan | 35.9 | 50 | 60 | 60 | 40 | 65 | | | | | | | |
| monooleate | | | | | | | | | | | | | |
| Sorbitan | | | | | | | 42.5 | 50 | 60 | 60 | 65 | | |
| sesquioleate | | | | | | | | | | | | | |
| Glycerin | | | | | | | | | | | | 46.5 | |
| monooleate | | | | | | | | | | | | | |
| Glycerol | | | | | | | | | | | | | 25 |
| dioleate | | | | | | | | | | | | | |
| Phosphatidyl | 46.2 | | | 35 | 48 | | 50.8 | | | 30 | | 33.5 | |
| choline | | | | | | | | | | | | | |
| Phosphatidyl | | 42.5 | | | | 25 | | 42.5 | | | 25 | | 60 |
| ethanolamine | | | | | | | | | | | | | |
| Phosphatidyl | | | 32.5 | | | | | | 32.5 | | | | |
| serine | | | | | | | | | | | | | |
| Triglyceride | | 7.5 | | | | | | 7.5 | | | | | |
| Retinyl | | | 7.5 | | | | | | 7.5 | | | | |
| palmitate | | | | | | | | | | | | | |
| Tocopherol | 12.8 | | | 5 | | | | | | 10 | | 14.4 | |
| acetate | | | | | | | | | | | | | |
| Benzyl | | | | | 7 | | | | | | | | 10 |
| benzoate | | | | | | | | | | | | | |
| Cholesterol | | | | | | 5 | 6.7 | | | | 5 | | 5 |
| Ethanol | 5.1 | | | | 5 | 5 | | | | | 5 | 5.8 | |

| | |
|---|---|
| Form in | Liquid Crystal |
| aqueous phase | |

### Comparative Examples 14 to 20

### Preparation of Pharmaceutical Compositions Devoid of Anionic Anchoring Agent

Liquid crystal formers, phospholipids, liquid crystal hardeners and cationic pharmacologically active substances were mixed at the weight ratios given in Table 4, below, optionally in a solvent.

In Comparative Examples 14 to 20, the substances were homogeneously in a water bath maintained at 20-75° C. using a homogenizer (PowerGen model 125, Fisher) for 0.5-3 hrs at 1000-3000 rpm. The resulting lipid solutions were left at room temperature to come to thermal equilibrium at 25° C., followed by adding each of the pharmacologically active substances dutasteride, leuprolide, exenatide, tamsulosin, and entecavir thereto. Then, the substances were homogenized using a homogenizer at 1000-3000 rpm for about 5-30 mins to afford pharmaceutical compositions in a solution phase.

TABLE 4

| | Comparative Example | | | | | | |
|---|---|---|---|---|---|---|---|
| (Unit: mg) | 14 | 15 | 16 | 17 | 18 | 19 | 20 |
| Dutasteride | 0.5 | | | | | | |
| Leuprolide | | 3.75 | | | | 3.75 | |
| Exenatide | | | 0.13 | | | | |
| Tamsulosin | | | | 0.2 | | | |
| Entecavir | | | | | 2.3 | | 2.3 |
| Sorbitan monooleate | 50.5 | 35 | | 30 | | | |
| Sorbitan sesquioleate | | | 45 | | 35 | | |
| Glycerin monooleate | | | | | | 45 | |
| Glycerin dioleate | | | | | | | 50.5 |
| Phosphatidylcholine | 32 | 45 | | 40 | 55 | 35 | |
| Phosphatidylethanol- | | | 40 | | | | 32 |
| amine | | | | | | | |
| Tocopherol acetate | 12.5 | 15 | | 20 | 10 | 15 | 12.5 |
| Cholesterol | | | 15 | 5 | | | |
| Ethanol | 5 | 5 | | 5 | | 5 | 5 |

| | |
|---|---|
| Form in aqeous phase | Liquid Crystal |

### Comparative Examples 21 and 22

For the formulation of Comparative Example 21, 3.75 mg of leuprolide was added with 1 mL of physiological saline, followed by homogenization at room temperature.

The formulation of Comparative Example 22 was prepared by dissolving 2.3 mg of entecavir in 1 mL of physiological saline at room temperature.

### Experimental Example 1

### Assay for In Vitro Safety

A cytotoxic test was carried out using an extraction colony assay to examine the anionic anchoring agents of the present invention for in vitro safety.

In 18 mL of Eagle's Minimal Essential Media (EMEM) supplemented with 10% fetal bovine serum was extracted 2 g of each of the compositions of Examples 2, 7 and 16, and Comparative Examples 5, 7 and 12. L929 cells (mouse fibroblast, American Type Culture Collection) were seeded at a density of 1×10² cells/well into 6-well plates, and stabilized for 24 hrs at 37° C. in a 5% CO₂ humidified incubator. The extracts were diluted in EMEM (0, 5, 25, 50%) and then placed in an amount of 2 mL/well in contact with the stabilized L929 cells.

After incubation for 7 days at 37° C. in a 5% CO₂ humidified incubator, the cells were fixed with a 10% formalin solution and stained with a Giemsa solution to count colonies. The results are summarized in Table 5, below.

TABLE 5

| Extract Medium | Relative Colony Formulation Rates (%)* | | | | | |
|---|---|---|---|---|---|---|
| (v/v)%** | Ex. 2 | Ex. 7 | Ex. 16 | C. Ex. 5 | C. Ex. 7 | C. Ex. 12 |
| 0% Medium | 100.0 | 100.0 | 100.0 | 100.0 | 100.0 | 100.0 |
| (control) | | | | | | |
| 5% Medium | 100.0 | 98.4 | 71.4 | 97.6 | 96.7 | 77.1 |
| 25% Medium | 68.4 | 74.5 | 23.8 | 70.7 | 71.9 | 26.2 |
| 50% Medium | 20.0 | 14.3 | 10.6 | 17.8 | 21.5 | 9.2 |

*Relative colony formation rates (%) = Number of Colonies on Test Medium/Number of Colonies on 0% Medium × 100 (%)
**Extract Medium % = Extract Medium/(Diluted Medium + Extract Medium) × 100 (%)

As is understood from data of Table 5, similar cell growth rates were observed between Example 2 and Comparative Example 7 in each of the diluted media (5%, 25%, and 50%) between Example 7 and Comparative Example 5, and between Example 16 and Comparative Example 12, indicating that the anionic anchoring agents of the present invention have no negative influence on safety in vivo.

### Experimental Example 2

### Assay for In Vivo Biodegradability

The compositions of the present invention were evaluated for in vivo biodegradability as follows.

Each of the compositions of Examples 2 and 13 was subcutaneously injected at a dose of 400 mg into the back of 6 SD rats (male) 9 weeks old, with an average bodyweight of 300 g, and monitored for a predetermined period of time. For comparison, the compositions of Comparative Examples 1 and 7 were tested in the same manner. The injection sites were photographed 2 weeks and 4 weeks after injection, and are shown in FIG. 2.

As can be seen in FIG. 2, the compositions of Examples 2 and 13 were observed to be biodegraded to the same degree as those of Comparative Examples 1 and 7, which thus indicates no influences of the anionic anchoring agents of the present invention on biodegradability.

### Experimental Example 3

### In Vivo Test for Sustained Release of Leuprolide

Drug release behaviors from the pharmaceutical compositions of the present invention were examined in vivo in the following test.

Using a disposable syringe, each of the pharmaceutical compositions of Example 21 and Comparative Example 15 was subcutaneously injected at a leuprolide acetate dose of 12.5 mg/kg (corresponding to a 28-day dose for humans) into the back of 6 SD rats (male), 9 weeks old, with an average body weight of 300 g. For comparison of PK profiles (pharmaceokinetic profiles, the composition of Comparative Example 21, which is a typical injection was injected at a leuprolide dose of 0.45 mg/kg (corresponding to a 1-day dose for humans) in the same manner.

Leuprolide acetate concentrations in plasma samples taken from the SD rats were monitored for 28 days using LC-MS/MS (liquid chromatography-mass spectrometry) to analyze PK profiles (pharmacokinetic profiles).

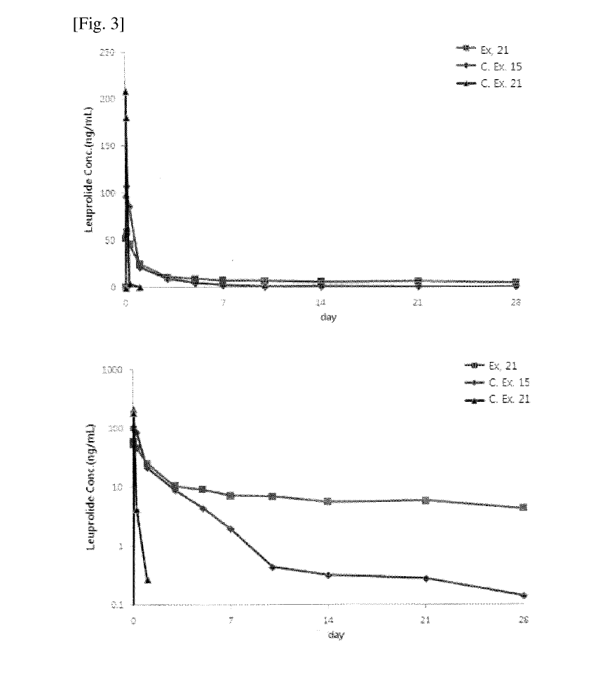

The PK profiles in the SD rats are shown in FIG. 3. As can be seen, the composition of Comparative Example 15 was about 2-fold higher in initial burst concentration and released the active substance at a slightly lower concentration for about 10 days after subcutaneous injection, compared to that of Example 21.

The initial burst concentration of Comparative Example 21 was about 5-fold higher than that of Example 21, but without sustained release after subcutaneous injection.

Consequently, the composition of Example 21 containing an anionic anchoring agent was observed to allows for excellent sustained release as demonstrated by the stable PK profile (pharmacokinetic profile) in which the drug was released for about 28 days or longer at a concentration sufficient to guarantee an efficient pharmaceutical effect, with a significant reduction of the initial burst concentration, thanks to the ionic interaction between the anionic anchoring agent and the cationic pharmacologically active substance partially or in its entirety.

Particularly characterized by a significant improvement in the initial burst, which is the drawback of conventional sustained-release formulations, the compositions of the present invention can give ideal PK profiles to guarantee sustained release for a desired period of time (means of measurements taken of 6 rats are plotted in the upper graph of FIG. 3, and expressed as calculated values of logarithm in the lower graph of FIG. 3 to examine a difference in drug plasma concentration at the late phase).

### Experimental Example 4

### In Vivo Test for Sustained Release of Entecavir

Drug release behaviors from the pharmaceutical compositions of the present invention were examined in vivo in the following test.

Using a disposable syringe, each of the pharmaceutical compositions of Example 24 and Comparative Example 18 was subcutaneously injected at an entecavir dose of 5.6 mg/kg (corresponding to a 7-day dose for humans) into the back of 6 SD rats (male), 9 weeks old, with an average body weight of 300 g. For comparison of PK profiles (pharmaceokinetic profiles, the pharmaceutical composition of Comparative Example 22, which is a typical injection, was injected at an entecavir dose of 0.2 mg/kg (corresponding to a 1-day dose for humans) in the same manner.

Entecavir concentrations in plasma samples taken from the SD rats were monitored for 28 days using LC-MS/MS to draw PK profiles (pharmacokinetic profiles).

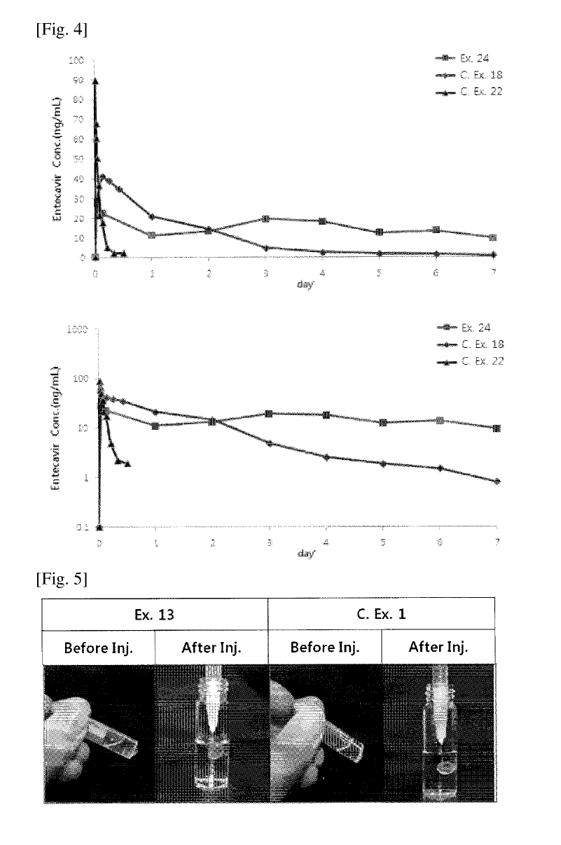

The PK profiles in the SD rats are shown in FIG. 4. As can be seen, the composition of Comparative Example 18 was about 1.5-fold higher in initial burst concentration, compared to that of Example 24, and released the active substance for about 3 days after subcutaneous injection.

The initial burst concentration of Comparative Example 22 was about 3-fold higher than that of Example 24, but without sustained release after subcutaneous injection.

Consequently, the composition of Example 24 containing an anionic anchoring agent was observed to allows for excellent sustained release as demonstrated by the stable PK profile (pharmacokinetic profile) in which the drug was released for about 7 days or longer at a concentration sufficient to guarantee an efficient pharmaceutical effect, with the significant reduction of the initial burst concentration, thanks to the ionic interaction between the anionic anchoring agent and the cationic pharmacologically active substance partially or in its entirety (means of measurements taken of 6 rats are plotted in the upper graph of FIG. 4, and are expressed as calculated values of logarithm in the lower graph of FIG. 4 to examine a difference in drug plasma concentration at the late phase).

### Experiment Example 5

### Formation of Liquid Crystal in Aqueous Fluid

The composition of the present invention was evaluated for ability to form liquid crystal in an aqueous phase as follows. After being loaded into syringes, compositions of Example 13 and Comparative Example 1 were dripped into 2 g of PBS (pH 7.4, and the results are shown in FIG. 5.

Both the compositions of Example 13 and Comparative Example 1 were observed to exist as a lipid liquid phase in the absence of aqueous fluid before injection, but formed into liquid crystal after exposure to aqueous fluid. Therefore, the anionic anchoring agent for enhancing sustained release in accordance with the present invention had no influences on the formation of liquid crystals.

Compositions of Examples 13 and Comparative Example 1 are in the form of a liquid phase, but rapidly form into liquid crystals contributing to a sustained release effect in the presence of aqueous fluid, so that they can be applied to the sustained release formulation of pharmaceutical agents.

Within the liquid crystals, there are a great number of bicontinuous water channels of nano size (below 20 nm) that resemble the Moebius strip. The water channels are surrounded with bicontinuous lipid layers. Thus, once a lipid composition forms into a liquid crystal in a semi-solid phase, a pharmacologically active substance can be released from the liquid crystal structure only after it has passed through numerous water channels and lipid layers, which enhances the sustained release effect of a pharmacologically active substance.

1. A sustained-release lipid pre-concentrate, comprising:
a) at least one liquid crystal former;
b) at least one neutral phospholipid;
c) at least one liquid crystal hardener; and
d) at least one anionic anchoring agent,
wherein the sustained-release pre-concentrate exists as a lipid liquid phase in the absence of aqueous fluid and forms into a liquid crystal upon exposure to aqueous fluid.

2. The sustained-release lipid pre-concentrate of claim 1, wherein the liquid phase former is selected from the group consisting of sorbitan unsaturated fatty acid ester, monoacyl glycerol, diacyl glycerol, and a combination thereof.

3. The sustained-release lipid pre-concentrate of claim 2, wherein the sorbitan unsaturated fatty acid ester has two or more —OH (hydroxyl) groups in the polar head.

4. The sustained-release lipid pre-concentrate of claim 2, wherein the sorbitan unsaturated fatty acid ester is selected from the group consisting of sorbitan monooleate, sorbitan monolinoleate, sorbitan monopalmitoleate, sorbitan monomyristoleate, sorbitan sesquioleate, sorbitan sesquilinoleate, sorbitan sesquipalmitoleate, sorbitan sesquimyristoleate, sorbitan dioleate, sorbitan dilinoleate, sorbitan dipalmitoleate, sorbitan dimyristoleate, and a combination thereof.

5. The sustained-release lipid pre-concentrate of claim 2, wherein the sorbitan unsaturated fatty acid ester is selected from the group consisting of sorbitan monooleate, sorbitan monolinoleate, sorbitan monopalmitoleate, sorbitan monomyristoleate, sorbitan sesquioleate, and a combination thereof.

6. The sustained-release lipid pre-concentrate of claim 2, wherein the monoacyl glycerol has a polar head consisting of glycerine, with a fatty acid tail attached thereto via an ester bond.

7. The sustained-release lipid pre-concentrate of claim 2, wherein the diacyl glycerol has a polar head consisting of glycerine, with two fatty acid tails attached thereto via respective ester bonds, said two fatty acid tails being the same or different from each other.

8. The sustained-release lipid pre-concentrate of claim 2, wherein the fatty acid groups attached to the monoacyl glycerol or the diacyl glycerol via ester bonds contains 4 to 30 carbon atoms, and is selected from the group consisting of palmitic acid, palmitoleic acid, lauric acid, butyric acid, valeric acid, caproic acid, enanthic acid, caprylic acid, pelargonic acid, capric acid, myristic acid, myristoleic acid, stearic acid, arachidic acid, behenic acid, lignoceric acid, cerotic acid, linolenic acid, alpha-linolenic acid (ALA), eicosapentaenoic acid (EPA), docosahexaenoic acid (DHA), linoleic acid (LA), gamma-linoleic acid (GLA), dihomo gamma-linoleic acid (DGLA), arachidonic acid (AA), oleic acid, vaccenic acid, elaidic acid, eicosanoic acid, erucic acid, nervonic acid, and a combination thereof.

9. The sustained-release lipid pre-concentrate of claim 2, wherein the monoacyl glycerol is selected from the group consisting of glycerol monobutyrate, glycerol monobehenate, glycerol monocaprylate, glycerol monolaurate, glycerol monomethacrylate, glycerol monopalmitate, glycerol monostearate, glycerol monooleate, glycerol monolinoleate, glycerol monoarchidate, glycerol monoarchidonate, glycerol monoerucate, and a combination thereof, and the diacyl glycerol is selected from the group consisting of glycerol dibehenate, glycol dilaurate, glycerol dimethacrylate, glycerol dipalmitate, glycerol distearate, glycerol dioleate, glycerol dilinoleate, glycerol dierucate, glycerol dimyristate, glycerol diricinoleate, glycerol dipalmitoleate, and a combination thereof.

10. The sustained-release lipid pre-concentrate of claim 2, wherein the monoacyl glycerol is glycerine monooleate (GMO) and the diacyl glycerol is glycerine dioleate (GDO).

11. The sustained-release lipid pre-concentrate of claim 1, wherein the neutral phospholipid is selected from the group consisting of phosphatidylcholine, phosphatidylethanolamine, phosphatidylinositol, sphingomyelin, and a combination thereof, having saturated or unsaturated carbon atoms in the range of 4 to 30.

12. The sustained-release lipid pre-concentrate of claim 1, wherein the liquid crystal hardener is free of an ionizable group and its hydrophobic moiety has a triacyl group with 15 to 40 carbon atoms or a carbon ring structure.

13. The sustained-release lipid pre-concentrate of claim 1, wherein the liquid crystal hardener is selected from the group consisting of triglyceride, retinyl palmitate, tocopherol acetate, cholesterol, benzyl benzoate, ubiquinone, and a combination thereof.

14. The sustained-release lipid pre-concentrate of claim 1, wherein the liquid crystal hardener is selected from the group consisting of tocopherol acetate, cholesterol, and a combination thereof.

15. The sustained-release lipid pre-concentrate of claim 1, wherein the anionic anchoring agent comprises a polar head and a hydrophobic moiety, said polar head containing at least one selected from the group consisting of a carboxylate, a phosphate, a sulfate or a sulfonate, said hydrophobic moiety containing 4 to 40 carbon atoms.

16. The sustained-release lipid pre-concentrate of claim 15, wherein the anionic anchoring agent with the carboxylate in the polar head is selected from the group consisting of palmitic acid, palmitoleic acid, lauric acid, butyric acid, valeric acid, caproic acid, enanthic acid, caprylic acid, pelargonic acid, capric acid, myristic acid, myristoleic acid, stearic acid, arachidic acid, behenic acid, lignoceric acid, cerotic acid, linolenic acid, alpha-linolenic acid (ALA), eicosapentaenoic acid (EPA), docosahexaenoic acid (DHA), linoleic acid (LA), gamma-linoleic acid (GLA), dihomo gamma-linoleic acid (DGLA), arachidonic acid (AA), oleic acid, vaccenic acid, elaidic acid, eicosanoic acid, erucic acid, nervonic acid, benzoic acid, sorbic acid, pamoic acid, lipoic acid, and a combination thereof.

17. The sustained-release lipid pre-concentrate of claim 15, wherein the anionic anchoring agent with the carboxylate in the polar head is selected from the group consisting of caprylic acid, capric acid, stearic acid, oleic acid, linolenic acid, benzoic acid, sorbic acid, lipoic acid, and a combination thereof.

18. The sustained-release lipid pre-concentrate of claim 15, wherein the anionic anchoring agent with the phosphate in the polar head is selected from the group consisting of phosphatidyl serine, phosphatidyl glycerine, phosphatidic acid, and a combination thereof.

19. The sustained-release lipid pre-concentrate of claim 15, wherein the anionic anchoring agent with the sulfate in the polar head is selected from the group consisting of lauryl sulfate, dodecyl sulfate, cholesteryl sulfate, and a combination thereof.

20. The sustained-release lipid pre-concentrate of claim 15, wherein the anionic anchoring agent with the sulfonate in the polar head is selected from the group consisting of benzene sulfonate, dodecyl benzene sulfonate, and a combination thereof.

21. The sustained-release lipid pre-concentrate of claim 1, wherein a weight ratio of a) to b) ranges from 10:1 to 1:10.

22. The sustained-release lipid pre-concentrate of claim 1, wherein a weight ratio of a)+b) to c) ranges from 1,000:1 to 1:1.

23. The sustained-release lipid pre-concentrate of claim 1, wherein a weight ratio of a)+b)+c) to d) ranges from 5,000:1 to 5:1.

24. A pharmaceutical composition, comprising:
the sustained-release lipid pre-concentrate of any one of claims 1 to 23; and
e) at least one cationic pharmacologically active substance,
wherein the anionic anchoring agent of the sustained-release pre-concentrate enhances the sustained release of the cationic pharmacologically active substance by forming an ionic bond with the cationic pharmacologically active substance.

25. The pharmaceutical composition of claim 24, wherein the cationic pharmacologically active substance is selected from the group consisting of pharmacologically active substance having at least one structure of a primary amine, a secondary amine, a tertiary amine, an aromatic amine, a sulfonium, an iodonium, an ammonium, a phosphonium, a pyridinium, a thiazolinium, an imidazolinium, a sulfoxonium, an isothiouronium, an azetidinium or a diazonium, a pharmaceutically acceptable salt thereof, and a combination thereof.

26. The pharmaceutical composition of claim 24, wherein the cationic pharmacologically active substance is selected from the group consisting of leuprolide, triptorelin, goserelin, nafarelin, buserelin, histrelin, deslorelin, meterelin, gonadrelin, entecavir, anastrozole, rivastigmin, acapodene, abiraterone, tibolone, fentanyl, tacrolimus, methotrexate, tamsulosin, dutasteride, finasteride, solifenacin, tadalafil, donepezil, olanzapine, risperidone, aripiprazole, naltrexone, varenicline, ropinirole, latanoprost, olopatadine, progesterone, ketotifen, montelukast, human growth hormone, tramadol, diazepam, diclofenac, pilocarpine, levocabastine, timolol, betaxolol, carteolol, levobunolol, epinephrine, dipivefrine, clonidine, apraclonidine, indomethacin, acyclovir, testosterone, statin, nifedipine, voriconazole, clotrimazole, ketoconazole, fulvestrant, fibrate, octreotide, estradiol, cortisone, progesterone, amphotericin B, chlorhexidine, corticosteroid, cyclosporine A, desmopressin, somatostatin, calcitonin, oxytocin, vasopressin, follitropin-alpha or beta, thyrotropin alpha, secretin, bradykinin, hypotensive tissue hormone, insulin or insulin derivatives, interferon, tuftsin, magainin, indolicidin, protegrin, polymyxin, gramicidin, vapreotide, exenatide, liraglutide, CJC-1131, AVE010, LY548806, TH-0318, BIM 51077, degarelix, glucagon, defensin, histatin, a pharmaceutically acceptable salt thereof, and a combination thereof.

27. The pharmaceutical composition of claim 24, wherein the cationic pharmacologically active substance is selected from the group consisting of leuprolide, triptorelin, goserelin, nafarelin, buserelin, histrelin, deslorelin, meterelin, gonadrelin, a pharmaceutically acceptable salt thereof, and a combination thereof.

28. The pharmaceutical composition of claim 24, wherein a weight ratio of a)+b)+c)+d) to e) ranges from 10,000:1 to 2:1.

29. The pharmaceutical composition of claim 24, being formulated into a dosage form selected from among an injection, a ointment, a gel, a lotion, a capsule, a tablet, a solution, a suspension, a spray, an inhalant, an eye drop, an adhesive, and a plaster and pressure sensitive adhesive.

30. The pharmaceutical composition of claim 24, wherein the dosage form is an injection.
